(19) United States
(12) Patent Application Publication
(10) Pub. No.: US 2009/0209913 A1
(43) Pub. Date: Aug. 20, 2009

(54) Trocar for thoracic surgery

(75) Inventor: Danilo Ferrari, Arezzo (IT)

Correspondence Address: POLLACK, P.C., THE CHRYSLER BUILDING, 132 EAST 43RD STREET, SUITE 760, NEW YORK, NY, 10017, US

(73) Assignee: Elenor S.r.l.

(21) Appl. No.: 12/075,886

(22) Filed: Mar. 14, 2008

(30) Foreign Application Priority Data
Mar. 14, 2007 (IT) FI2007A000060

(51) Int. Cl. A61B 17/34

(52) U.S. Cl. 604/165.04

(57) ABSTRACT

A trocar for thoracic surgery, comprising a guide duct for surgical instruments adapted to be inserted in a body opening of access to the thoracic cavity. The duct is formed by a plurality of sectors arranged about its axis and articulated to a support frame. A sector expander is associated with the duct for opening the sectors from an initial insertion position in the body opening to a final maximum expansion position in which they are moved away from the axis to a greater extent than in the configuration assumed in the initial position. The expander includes a control ring nut movably connected to the sectors and the support frame such that rotation of the ring nut in one direction, results in the sectors movement away from the axis. Associated with the ring nut is a device for locking the rotation in the direction opposite that of the expansion direction. The device or rotation lock comprises a device for temporarily removing the rotation hindrance of the ring nut in the direction opposite the expansion direction, whereby the sectors can move backwards to the initial position.

### FIELD OF THE INVENTION

The present invention relates generally to medical devices and, more particularly, devices for use in laparoscopic surgery and the like.

### BACKGROUND OF THE INVENTION

Trends in thoracic surgery have increasingly been toward the development of surgical techniques that reduce invasiveness, especially where cardiac intervention is required. This has been made possible by an endoscopic surgery technique developed mainly for abdominal cavity operations, the so-called “laparoscopic surgery”. Laparoscopic surgery allows the use of devices, called “trocars”, that generate and maintain access paths for the various surgical instruments into the body cavity where the operation takes place. In practice, the trocars are formed by a cannula having a side abutment for engaging the walls of the body opening made by the surgeon to access the internal cavity.

The trocars used for laparoscopic surgery are different from the trocars used in thoracic surgery. In the first case, in fact, it is normal practice to blow gas into the abdominal cavity, so to dilate and stretch out its tissues in order to facilitate internal vision. For this reason, the trocars for laparoscopic surgery are equipped both with valves that prevent the gas from escaping therefrom, and with anchoring means for securing the trocar to the side walls of the opening in which the trocars are inserted and opposing the expulsion thrust caused by the presence of gas in the cavity.

In thoracic surgery there is no need to insert gas inside the cavity to be operated (or at least there is no need to keep such cavity pressurised), and moreover the overall tissue thickness to be crossed in order to reach the cavity is lower than the tissue thickness to be crossed when the abdominal cavity has to be reached. For this reason, the trocars for thoracic surgery are structurally much simpler and compact with respect to the trocars for laparoscopic surgery.

In order to minimise, as much as possible, the invasiveness of the incision in which the trocar is inserted, the trocars are made of small dimensions and are subsequently enlarged (by virtue of the tissue elasticity) by means of a retractor.

The surgeon is thus obliged to carry out the trocar insertion in different steps, a first step in which a retractor is inserted in the cavity access incision, a second step in which the incision is enlarged due to the action of the retractor and a third step in which the trocar is inserted while the retractor is extracted.

### OBJECTS AND SUMMARY OF THE INVENTION

Accordingly, it is an object of the present invention to provide a trocar for thoracic surgery that simplifies its insertion into an access incision of a patient's thoracic cavity.

Another important object of the invention is to provide a trocar for thoracic surgery of the above mentioned type that is structurally simple and easy to use.

These and other objects, which will be clearer below, are attained by a trocar for thoracic surgery comprising a guide duct for surgical instruments, the duct being adapted to be inserted in a body opening for access to the thoracic cavity, and formed by a plurality of sectors arranged around its axis and articulated to a support frame, sector expansion means being associated with the duct for mutually displacing the sectors from an initial insertion position in the body opening to a final maximum expansion position in which they are moved away from said axis to a greater extent than in the configuration assumed in the initial position, said expansion means comprising a control ring nut movably connected to the sectors and to the support frame, such that a rotation of the ring nut in one direction, called the expansion direction, results in a movement of the sectors away of the axis, means for locking the rotation in the opposite direction with respect to the expansion direction being associated with the ring nut, the rotation locking means comprising a temporary removal device of the rotation hindrance of the ring nut in the direction opposite the expansion direction, whereby the sectors can move backward to the initial position.

### BRIEF DESCRIPTION OF THE DRAWINGS

A specific, illustrative trocar, according to the present invention, is described below with reference to the accompanying drawings, in which:

FIG. 1 is an exploded perspective view of a trocar, according to one aspect of the present invention;

FIG. 2 is an axonometric view of the trocar set forth in FIG. 1 with expansion sectors in a starting or initial insertion position in a patient's body opening for accessing the thoracic cavity;

FIG. 3 is a sectional view of the trocar shown in FIG. 2;

FIG. 4 is an axonometric view of the trocar shown in FIG. 1 with the expansion sectors in an ending or final, maximum expansion position;

FIG. 5 is a sectional view of the trocar illustrated in FIG. 4;

FIG. 6 is a sectional view of the trocar shown in FIGS. 1, 2 and 4 with the expansion sectors in the starting position, and with a ratchet mechanism for keeping the sectors in stable intermediate expansion positions between their starting and ending positions;

FIG. 7 is an enlarged partial sectional view of the trocar and ratchet mechanism illustrated in FIG. 6;

FIG. 8 illustrates a portion of the trocar shown in FIG. 6 along a section analogous to that of FIG. 7 with the ratchet mechanism when the sectors are in the ending or final, maximum expansion position;

FIG. 9 shows a longitudinal section of the trocar set forth in FIGS. 1 and 2, with the expansion sectors in the starting position, and with an accessory element for locking the expansion sectors upon insertion in the patient's body opening;

FIG. 10 is a sectional view of the trocar shown in FIG. 1 with the expansion sector locking element according to FIG. 9; and

FIG. 11 is an axonometric view of the trocar illustrated in FIGS. 1 and 2 with the expansion sectors in the starting position, and with a key for facilitating movement of the control ring nut when positioning the expansion sectors.

The same numerals are used throughout the drawing figures to designate similar elements. Still other objects and advantages of the present invention will become apparent from the following description of the preferred embodiments.

### DETAILED DESCRIPTION OF THE PREFERRED EMBODIMENTS

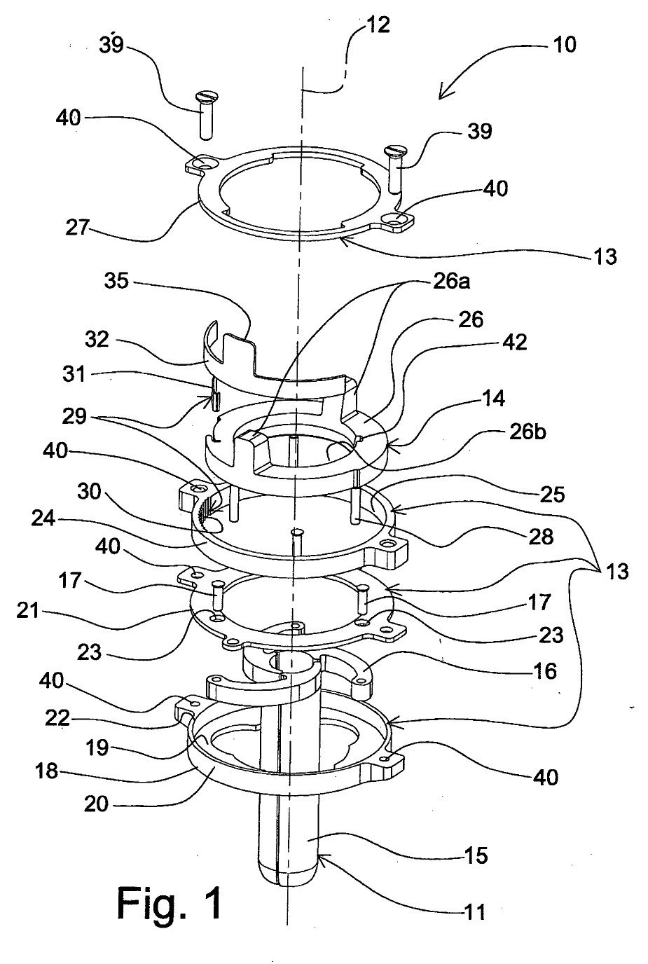

Referring now to the drawings and, more particularly, to FIGS. 1-11, there is shown generally a specific, illustrative trocar 10 for thoracic surgery, according to various aspects of the present invention. In one embodiment, illustrated generally in FIG. 1, the trocar comprises a guide duct T for surgical instruments configured for insertion in a patient's body opening for accessing to the thoracic cavity. Duct T includes a plurality of sectors 11 arranged about an axis 12 of the duct and articulated to a support frame 13.

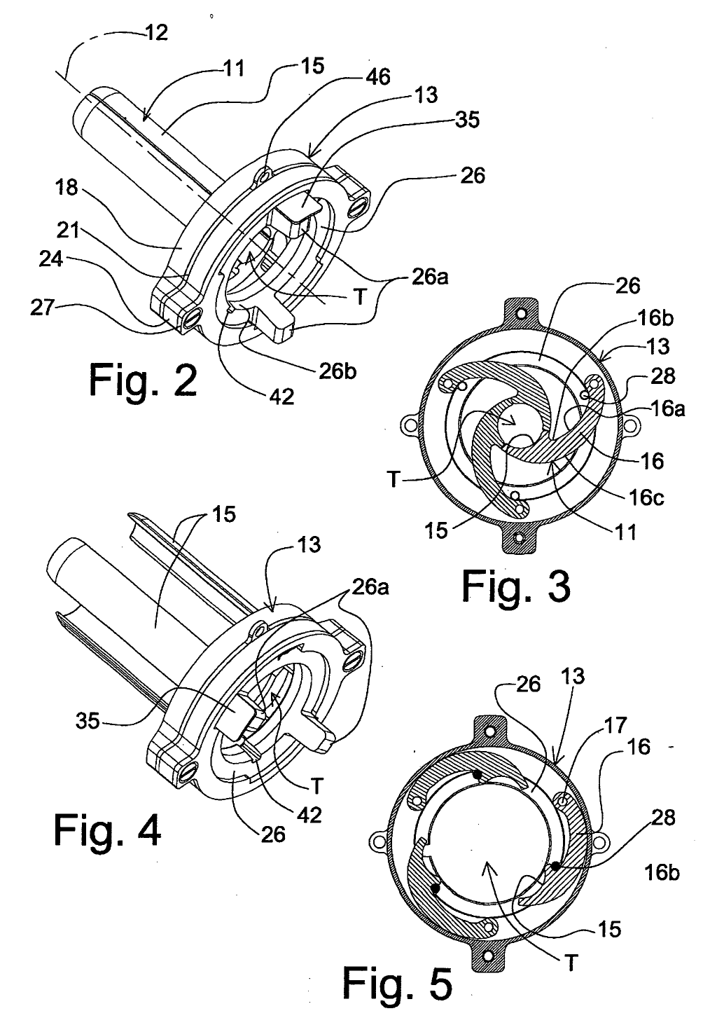

Associated with such duct T are expansion means 14 (described below) from an initial insertion position in the body opening (see FIGS. 2 and 3) to a final, maximum expansion position (see FIGS. 4 and 5) in which the sectors are moved away from the axis 12 to a greater extent than in the configuration assumed when they are in the initial position.

Each sector 11 comprises a longitudinal body 15 extending parallel to the axis 12. A preferably curved arm 16, connected at a free end to the frame 13 by means of a hinge pin 17, extends from one end of the longitudinal body 15 and on a plane orthogonal to the axis 12. It has to be noted that, when the sectors 11 are in their initial position, the longitudinal bodies 15 form a cylindrical duct closed along its own side surface.

In the present embodiment, the frame 13 comprises a first annular element 18 having an internal base 19, on which the arms 16 of the sectors 11 are slidingly housed, and a lateral containment wall 20 for the curved arms 16.

The frame 13 also comprises a second annular element 21, fixed to the upper edge 22 of the wall 20 of the first annular element 18. The hinge pins 17 for the arms 16 are connected to the second annular element 21 and, in particular, pins 17 are partially inserted in through holes 23 formed thereon. A third annular element 24 of the frame 13 is fixed to the second annular element 21 on the opposite side relative to the first annular element 18 and has an internal cylindrical side surface 25 that defines a rotational housing space for a control ring nut 26, for controlling the movement of the sectors 11. The control ring nut 26 is locked in the movement along the axis 12 between the second annular element 21 and a fourth annular element 27 fixed to the edge of the third annular element 24 on the opposite side with respect to the second annular element 21.

The expansion means 14, which provide spreading out or opening out of the sectors 11 comprise the control ring nut 26 and on the surface of the nut 26 facing towards the second annular element 21 pins 28 are provided that extend until they come into contact with the inner sides 16a, i.e. the concave sides facing towards the axis 12, of respective arms 16. Following a rotation of the control ring nut 26 in one direction, from hereafter referred to as expansion direction, the pins 28 are adapted to slide along the inner sides 16a in order to permit the opening movement of the sectors 11 from the initial insertion position (FIG. 2) in the body opening for accessing to the thoracic cavity, to the final, maximum expansion position (FIG. 4). On inner sides 16a, at the attachment end zone of the arms 16 to the longitudinal bodies 15, end stop abutments 16b are formed for the pins 28 when the sectors 11 are in the maximum expansion position.

The ring nut 26 has, on the surface opposite that where the pins 28 are formed, two diametrically opposed reliefs 26a that constitute grip elements for the surgeon's hand or abutment elements for an accessory element, such as a key C that allows the movement of the control ring nut, as shown in FIG. 13 and as will be better explained below.

Associated with the control ring nut 26 are means 29 for locking the rotation of the same ring nut in the direction opposite the expansion direction. In this embodiment, the means 29 for locking the rotation of the ring nut 26 are, for example, a ratchet mechanism interposed between the support frame 13 and the ring nut 26. In particular, the ratchet mechanism comprises a toothed portion 30 (with saw tooth shape), defined on the inner side surface 25 of the third annular element 24, and a pawl 31 elastically engageable with the toothed portion 30. The pawl 31 is for example formed by an elastic plate fixed at one end to the cylindrical side of the ring nut 26, the other end being free to slide on the toothed portion 30, in the direction permitted by the slope of the teeth.

The rotation locking means 29 moreover comprise a device 32 for temporarily removing the ring nut rotation hindrance that, with reference to the ratchet mechanism just described, is formed by a disengagement device of the pawl 31 from the toothed portion 30. As is clearly visible in FIGS. 7 and 8, the pawl 31 is arranged in a recess 33 on the cylindrical flank of the ring nut 26. The device 32 consists of a tongue 34, slidably arranged between the cylindrical flank of the ring nut 26 and the inner side surface 25 of the third annular element 24. Depending on the angular position taken along the cylindrical flank of the ring nut 26, the tongue 34 can be interposed between the free end of the elastic plate forming the pawl 31 and the toothed portion 30, thus permitting the disengagement of the pawl. The tongue 34 has a control appendage 35 that projects in the direction opposite the extension of the longitudinal bodies 15. The control appendage 35 is provided at one of the two reliefs 26a, and faces thereto.

It is noted how the width of the control appendage is greater than the width of the corresponding relief 26a.

In the maximum opening position of the trocar, or the final, maximum expansion position of the sectors 11, the trocar is in stable equilibrium and cannot be spontaneously closed due to the radial pressure exerted by the expanded tissues, while in the partial opening configurations, the ratchet mechanism prevents the spontaneous closure due to said pressure.

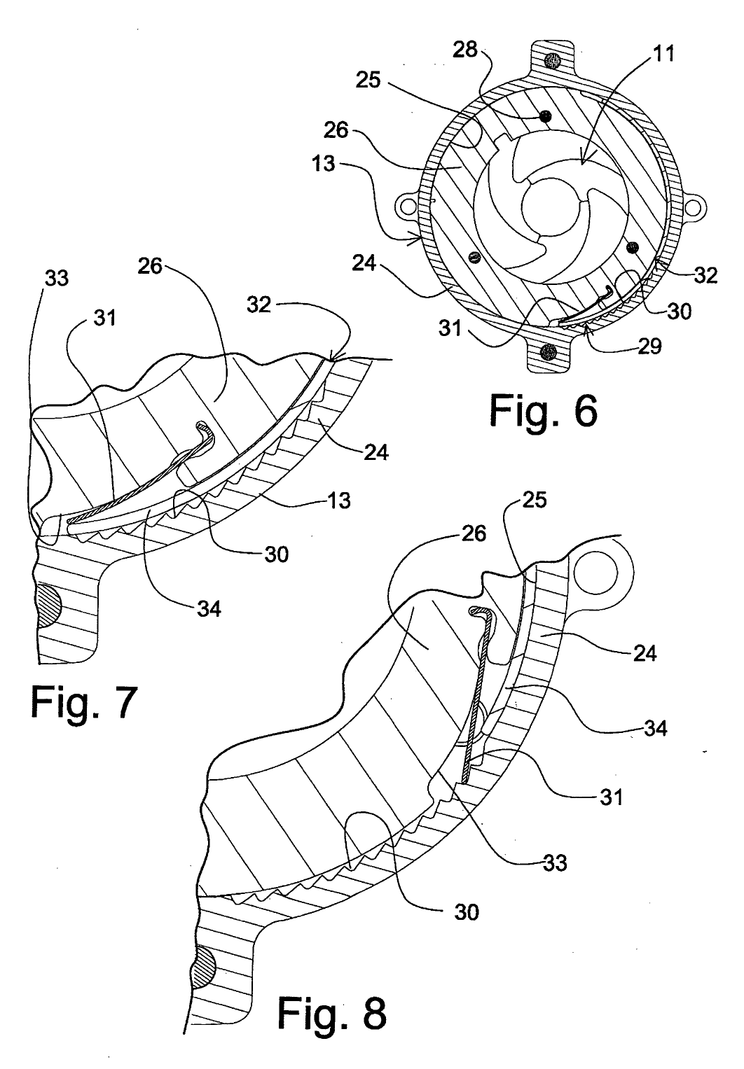

In order to close the trocar and bring the sectors 11 back into the initial insertion position in the opening made in the tissues to access the thoracic cavity, it is necessary to move the tongue 34. The tongue 34 can carry out a short rotation relative to the ring nut 13 by passing from the locking position (FIG. 8) to the release position (FIGS. 6 and 7). In the locking position, the elastic plate forming the pawl 31 is free to engage on the toothed portion 32 of the third annular element 24, while in the release position the tongue 34 causes the elastic plate to be bent, forcing it to abandon the grip on the toothed portion 31 and rearranging it inside the recess 33.

In order to expand the trocar, it is necessary to rotate the ring nut 26 in anticlockwise direction by operating on reliefs 26a by hand or with the suitable key C of FIG. 13. Since the control appendage 35 of the tongue 34 has a width greater than that of the corresponding relief 26a to which it faces, the right side of the control appendage 35 is first aligned with the right side of the relief 26a of the ring nut 26, bringing the tongue 34 from a release position, in which it is interposed between the pawl 31 and the toothed portion 30, to the locking position, in which the pawl 31 is free to engage on the saw teeth. A further rotation of the key (or hand) causes the pins 28 of the ring nut 26 to push the arms 16 towards the outside, with consequent moving away of the sectors 11 from the axis 12. The sectors will always remain in expanded state thanks to the ratchet mechanism, when in operation.

The key C has an end portion 36 adapted for being inserted in the hole 26b of the ring nut 26 and two side abutment portions 37 that cooperate with the control appendage 35 and the reliefs 26a. Moreover the key C has a hole 38 through which the surgeon distinguishes by the touch the grip side for the opening of the trocar from the grip side for the closing.

The annular elements forming the frame 13 are connected by threaded connections passing through the annular elements, such as for example a pair of opposing screws 39 passing through corresponding eyelets 40 formed peripherally on the annular elements 18, 21, 24 and 27.

The expansion of the sectors 11 is in practice monodirectional, as the three longitudinal bodies 15 are free to be moved away from the axis 12 but not to return back, by virtue of the ratchet mechanism. This is not a problem when the trocar is inserted in the patient's thorax, as sector expansion is opposed by the radial forces exerted by the elastic resistance of the patient tissues, but could constitute a drawback on the trocar insertion step, as a possible abutment contact could generate radial thrusts causing the sectors to expand before the insertion. Therefore, in order to avoid such risk, a further accessory element is provided, like an inserter tool U for preventing the expansion of the sectors 11 from the initial insertion position. This tool is usually associated with the trocar on the insertion step in the body opening and is subsequently removed before starting the expansion step.

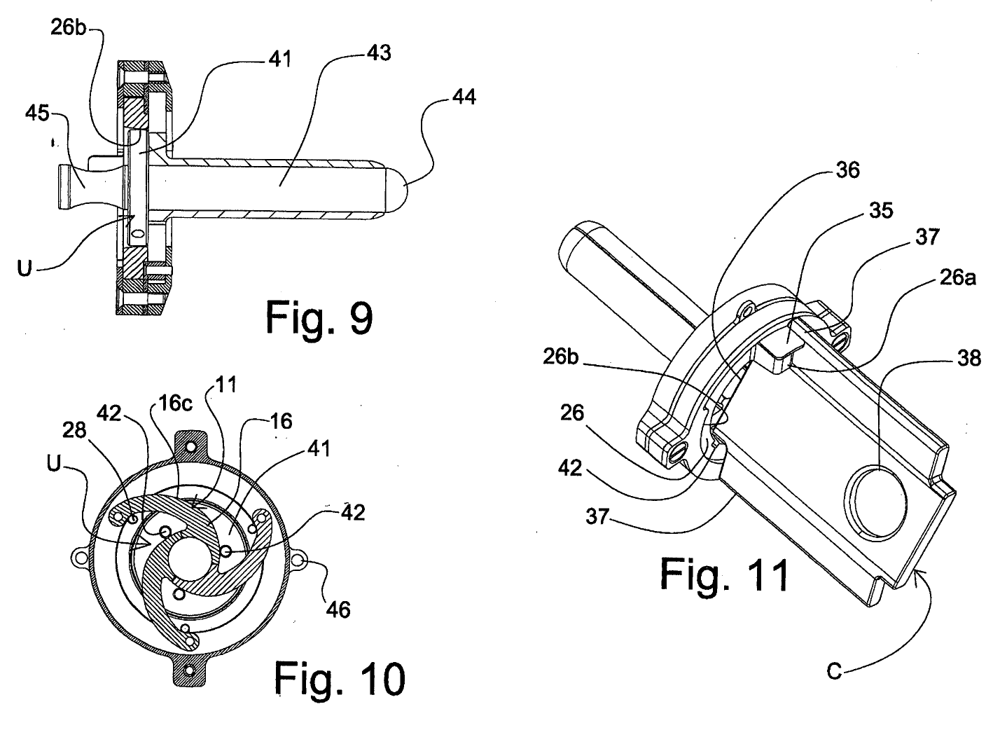

In particular, in this embodiment, such inserter tool U, visible in FIGS. 9 and 10, has a plate 41 with a shape complementary to the hole of the ring nut 26 and adapted to be inserted therein. Pins 42 are provided on the side of the plate 41 facing towards the sectors 11, each pin abutting against the external flank 16c of a respective arm 16, opposite the internal flank 16a on which the pins 28 of the ring nut 26 slide. The inserter tool U is secured to the ring nut 26 by means of a locking and positioning element (not shown in the drawings) projecting from the side of the plate 41 and adapted to be coupled with a groove 42 formed on the inner side surface of the ring nut 26. Advantageously, a rod-like portion 43 extends from the plate 41, in such a way to occupy the space defined between the longitudinal bodies 15 forming the sectors 11. The rod-like portion 43 projects from the duct T formed by sector 11 with one sphere-shaped end 44 that makes the insertion of the trocar in the body opening easier. On the opposite side, the plate 41 has a grip handle 45.

In order to prevent the trocar from slipping outside the opening made in the patient's thorax during the operation, the surgeon can secure the trocar to the thorax by means of fixing means, such as for example small rings 46 formed along the outside of the frame 13 and through which the surgeon can pass suture points for anchoring them to the patient's skin. Alternatively, the longitudinal bodies 15 forming the expansion sectors can be made with external surface with increased friction, by means of saw tooth corrugations or threads (elements not shown in the drawings for the sake of simplicity).

It is evident how the trocar described here is easily insertable in the body opening of access to the thoracic cavity, as during the insertion, the trocar sectors defining the access duct to the thoracic cavity have a very limited axial size. When the trocar is inserted in the thorax, the sectors can radially expand by enlarging the thoracic cavity access opening in order to permit the easy insertion of the surgical instruments.

In practice, a trocar and a retractor have been joined in a single instrument. Thus the number of tools to be used is reduced and the trocar insertion process simplified, entirely to the advantage of the surgeon who does not need to use additional instruments while the preliminary operating steps of the operation are reduced.

The trocar according to the invention can be subject to numerous modifications and variants, all being within the scope of the invention; moreover, all details can be replaced by other technically equivalent elements, without departing from the scope of the invention.

In practice, the materials used (provided that they are compatible with the specific use) as well as the size can be of any type according to technical requirements and the state of the art.

Various modifications and alterations may be appreciated based on a review of this disclosure. These changes and additions are intended to be within the scope and spirit of the invention as defined by the following claims.

What is claimed is:

1. A trocar for thoracic surgery, which comprises a guide duct for surgical instruments configured for insertion in a patient's body opening upon access to a thoracic cavity, wherein the duct is formed by a plurality of sectors arranged about its axis and articulated to a support frame, a sector expander being associated with the duct for mutually displacing the sectors from an initial insertion position in the body opening to a final, maximum expansion position in which they are moved away from the axis to a greater extent than in the configuration assumed in the initial position, the expander comprising a control ring nut movably connected to the sectors and the support frame, such that rotation of the ring nut in one direction results in a movement of the sectors away from the axis, a lock for preventing rotation in the direction opposite that of the one direction being associated with the ring nut, the rotation lock comprising a device for temporarily removing the rotation hindrance of the ring nut in the direction opposite the one direction, whereby the sectors can move backwards to the initial position.

2. The trocar set forth in claim 1, wherein the rotation lock of the control ring nut has a ratchet mechanism interposed between the frame and the control ring nut.

3. The trocar set forth in claim 2, wherein the ratchet mechanism comprises a toothed portion with a saw tooth profile formed on the frame, a pawl elastically associated with the control ring nut, one end of the pawl being free to slide on the toothed portion in the direction permitted by the slope of the teeth, the device for temporarily removing the rotation hindrance of the ring nut in the direction opposite the expansion direction comprising a disengagement device of the pawl from the toothed portion.

4. The trocar set forth in claim 3, wherein the frame internally defines a rotational housing space for the control ring nut, the toothed portion with saw tooth profile being formed on the inner side surface of the housing space for the control ring nut, the pawl being formed by an elastic plate with one end fixed to the cylindrical flank of the control ring nut and with the other end free to slide on the toothed portion in the direction permitted by the slope of the teeth, the device for disengaging the pawl from the toothed portion comprising a tongue slidably arranged between the cylindrical flank of the control ring nut and the inner side surface of the frame, the tongue being adapted to be interposed between the free end of the elastic plate forming the pawl and the toothed portion according to the angular position along the cylindrical flank of the control ring nut, thus permitting the disengagement of the pawl.

5. The trocar set forth in claim 4, wherein the control ring nut has at least one relief constituting either a grip element for the surgeon's hand or abutment element for a driving key of the ring nut, the at least one relief extending in the direction opposite the extension direction of the sectors, the disengagement tongue of the pawl of the ratchet mechanism having a control appendage provided at the at least one relief and facing the latter, the width of the control appendage being greater than the width of the corresponding the at least one relief.

6. The trocar set forth in claim 1, wherein each of the sectors comprises a longitudinal body that is extended in the same direction of the axis, an arm articulated at one free end to the frame by a hinge pin extending from one end of the longitudinal body, on a plane substantially orthogonal to the axis.

7. The trocar set forth in claim 6, wherein, when the sectors are in the initial insertion position, the longitudinal bodies form a cylindrical duct that is substantially closed along its own side surface.

8. The trocar set forth in claim 7, wherein each arm is curved with concavity turned towards the axis.

9. The trocar set forth in claim 8, wherein the frame is annular and surrounds the sectors, the frame comprising:
a first annular element having an inner base on which the arms of the sectors slidably abut, and a lateral containment side for the arms;
a second annular element, fixed on the upper edge of the side of the first annular element, to which hinge pins for the arms are bonded;
a third annular element fixed on the second annular element on the part opposite the first annular element, the third annular element having a cylindrical inner side surface that defines a rotational housing space for the control ring nut; and
a fourth annular element fixed on the edge of the third annular element on the side opposite the second annular element, the control ring nut being locked in movement along the axis between the second annular element and the fourth annular element.

10. The trocar set forth in claim 9, wherein the joining of the first, second, third and fourth annular element forming the frame is achieved by two opposing screws passing through corresponding eyelets made peripherally on the same annular elements.

11. The trocar set forth in claim 9, wherein the toothed portion of the ratchet mechanism is formed on the inner side surface of the third annular element of the frame.

12. The trocar set forth in claim 1, wherein the expander comprises pins that are extended from the surface of the control ring nut up to contact with the inner flanks of the arms, the pins being slidable on the flanks to allow the opening movement of the sectors from the initial insertion position in the body opening for accessing to the thoracic cavity to the final maximum expansion position.

13. The trocar set forth in claim 12, wherein on the inner flanks of the arms, at the attachment end zone of the arms to the longitudinal bodies forming the sectors, end stop abutments are formed for the pins when the sectors are in the maximum expansion position.

14. The trocar set forth in claim 1, further comprising a device for securing the trocar to the patient's thorax.

15. The trocar set forth in claim 14, wherein the securement device includes relatively small rings along the outside of the frame adapted to permit passage of suture points for anchoring to the patient's skin.

16. The trocar set forth in claim 1, further comprising an accessory element formed by an inserter tool adapted to lock the expansion of the sectors in the initial insertion position, the inserter tool being associable with the trocar body only in the insertion step in the body opening and being subsequently removed before beginning the expansion step.

17. The trocar set forth in claim 16, wherein the inserted tool has a plate countershaped to the hole of the control ring nut and fit for being inserted therein, the plate having, on the face turned towards the sectors, pins adapted to abut the external flanks, opposite the internal flanks on which the pins of the control ring nut slide, of respective arms, the inserter tool being locked on the control ring nut by a locking and positioning element projecting from the side of the plate and adapted to be coupled with a corresponding groove made on the inner side surface of the control ring nut.

18. The trocar set forth in claim 17, wherein a rod-like portion extends from the plate in the space defined between the longitudinal bodies forming the sectors, the rod-like portion projecting from the duct formed by these with a shaped end to make easier the insertion of the trocar in the body opening.

19. The trocar set forth in claim 1, further comprising an accessory element formed by a key that can be coupled with the control ring nut to control its rotation.

20. The trocar set forth in claim 19, wherein the key has an end portion engageable in the hole of the control ring nut, and at least one side abutment portion that interacts with the control appendage of the ratchet mechanism and with the at least one relief of the ring nut, the key having a member for distinguishing the grip side for the opening of the trocar from the grip side for the closing.
